(19) United States
(12) Patent Application Publication
(10) Pub. No.: US 2007/0264264 A1
(43) Pub. Date: Nov. 15, 2007

(54) METHOD AND COMPOSITION FOR CONFERRING IMMUNITY IN MAMMALS

(75) Inventors: David Evans, Custer, WA (US); F. Joseph Daugherty, Omaha, NE (US); David De Klyen, Mount Vernon, WA (US)

Correspondence Address: HALL, VANDE SANDE & PEQUIGNOT, LLP, 1636 R STREET N.W., THIRD FLOOR, WASHINGTON, DC, 20009, US

(73) Assignee: LA BELLE ASSOCIATES, INC., Bellingham, WA (US)

(21) Appl. No.: 11/468,466

(22) Filed: Aug. 30, 2006

Related U.S. Application Data
(60) Provisional application No. 60/712,453, filed on Aug. 30, 2005.

(51) Int. Cl. A61K 39/395; A61K 39/40; A61K 39/42; A61P 31/04; A61P 31/12

(52) U.S. Cl. 424/157.1; 424/130.1; 424/159.1

(57) ABSTRACT

A composition and method for immunization of mammals, containing antibodies from the eggs/egg yolks of chickens, or other suitable avian source, hyperimmunized against a selected pathogen(s) or immunogens, and a quantity of non-specific mammalian antibodies, such as those from commercial colostrum or serum products, which are combined to produce a composition that provides protection against the selected pathogen. Applications include viral, bacterial and parasitic infections.

### FIELD OF THE INVENTION

The present invention relates to compositions and processes for providing immune protection for mammalian species using a combination of avian and mammalian antibody sources.

### BACKGROUND OF RELATED ART

In mammalian species immunity to pathogens is transferred from mother to offspring via the placenta or colostrum. The mother transfers only those antibodies that were built up by her due to natural exposure or vaccinations. However, her level of transfer of antibodies is influenced by how recently exposure to particular pathogens occurred. If the maternal colostrum contains an insufficient quantity of antibodies specific for certain pathogens, the neonate will have a deficient level of immunity for those diseases.

In the case of mammals that transfer immunity via colostrum, a greater importance is placed on the ability of the neonate to suckle. A weak nursing neonate, or one that is removed from the mother immediately after birth, may not obtain a sufficient or minimum level of antibodies necessary for protection against invading pathogens. This is known as “failure of passive transfer.”

Specific antibody building via immunization of an avian species, such as chickens, is also well documented. However, the specificity and functionality of avian antibodies is a hindrance to their application in mammals. Avian-sourced antibodies (IgY) do not contain a constant (F_{c}) region that binds with the mammalian complement system. An important function of mammalian antibodies (IgG) is the ability of the constant (F_{c}) region to mediate effector functions such as the triggering of the complement cascade. The binding of an antibody to an antigenic determinant on an invading pathogen (e.g. the surface of a foreign cell) leads to a cascade of reactions that lyses the cell due to complement opsinization. The binding of complement proteins to the F_{c} units of antigen-antibody complexes triggers the cascade.

U.S. Pat. No. 4,748,018, issued to Stolle et al., describes the administration of avian antibodies to mammals to provide protection against oral and gastrointestinal infections. Specifically, Stolle et al. disclose a method of passive immunization, which comprises: (a) feeding a mammal a material having an enhanced antibody titer against an antigen, obtained from the egg of a domesticated fowl immunized against said antigen, until the mammal develops substantial tolerance to the antibodies; and (b) administering to the mammal an antibody obtained from a domesticated fowl immunized against said antigen. In other words, prior to the administration to the mammal of antibodies obtained from the domesticated fowl, the mammal is fed a material derived from eggs of a fowl immunized against the antigen. The need to feed the mammal material from immunized eggs prior to administration of the avian antibodies obviously means the method is of very limited utility against acute infections and diseases. Moreover, it is difficult, if not impossible, to apply the method of Stolle et al. to neonates.

Stolle et al. also disclose a composition for use in the above-described method, comprising: (a) a material having an enhanced antibody titer against a given mammalian antigen, obtained from the egg of a domesticated fowl immunized against said antigen; together with (b) a material having an enhanced antibody titer against said antigen obtained from the milk of a domesticated bovid immunized against said antigen. Such immunization for specific pathogens of a mammalian species (e.g. immunization of bovids to produce milk containing desired antibodies) is well documented, however, suffers from significant logistical complications relating to the cost of maintaining controlled herds as required by USDA and most other international regulatory agencies.

Such difficulties are also discussed in European Patents 0 930 316 and 0 914 831, (Mican et al.), in which the inventors that the immunization of dams (i.e. the production of antibodies from an immunized bovine source) is difficult for number of reasons:
a) antibodies present in blood at the time of immunization can partly or completely neutralize the administered antigens and thus to reduce or even eliminate the expected immunization effect;
b) relevant concentrations of specific antibodies persist in colostrum only during the first 24 to 48 hours after parturition and, consequently, the local passive immunity of the gastrointestinal tract is limited to the first three days of life; and
c) the methods of preparation of specific immunoglobulins from colostrum of immunized cows are rather sophisticated and expensive and the efficacy of the specific immunoglobulins is insufficient in calves suffering from gastrointestinal infections manifested by diarrhoea of various degrees of severity and other symptoms.
   Mican et al. also claim that attempts to treat calves with antibodies prepared from blood serum of immunized animals were unsuccessful, in part because orally administered serum immunoglobulins are rapidly degraded by gastric and intestinal enzymes. Despite these difficulties, and in keeping with conventional wisdom in the field, Mican et al. claim that the most effective protection is provided by administering compositions containing antibodies from immunized cows and/or hens (in combination with other substances, such as lactacidogenic bacteria).

Specifically, Mican et al. disclose oral products for the treatment of dogs and calves, comprising antibodies specific for bovine rotavirus, bovine coronavirus, enterotoxigenic strains of Escherichia coli and/or canine parvovirus, obtained from immunized cows (colostrum) or hens (egg yolk, combined with stabilized live lactacidogenic bacteria. The inventors theorize that the invention is effective because of the combination of the specific passive immunotherapy (i.e. bovine or hen antibodies from immunized sources) with the non-specific prophylactic effect of the probiotics (i.e. lactacidogenic bacteria).

Perhaps recognizing the difficulties with the generation and use of antibodies described by Mican et al., in U.S. Pat. No. 6,866,868 Lisonbee et al. describe methods and compositions comprising transfer factors from non-immunized mammalian and non-mammalian sources (e.g. bovine and avian sources derived from colostrum and egg, respectively). Although relatively poorly characterized and understood, transfer factors are less than 10,000 MW molecules that are readily absorbed in the digestive system, whereas antibodies are huge molecules >100,000 MW that generally aren't absorbed well.

Lisonbee et al. theorize that administering transfer factors from two different species provides protection against a broader range of pathogens than administration of transfer factor from a single species. Lisonbee et al. are not concerned with antibody dependent immunity to a specific pathogen, but rather boosting (triggering an immune reaction) immunity in general. Transfer factors are believed to stimulate or trigger cell-mediated immunity and are not known to cause the production and release of antibodies per se. They have no known functionality as humoral (circulating) immunity agents nor do they posses biological or molecular activity as antibodies.

In U.S. patent application Ser. No. 10/103,671, Lisonbee et al. describe a related invention directed to methods for generating and preparing the egg-derived transfer factor. The use of avian transfer factor is preferred to avian antibodies because: “[0028] Treatment of pathogenic infections in mammals with avian antibodies is typically not desirable, however, since the immune systems of mammals are likely to respond negatively to the large avian antibody molecules by eliciting an immune response to the antibodies themselves. Moreover, as mammalian immune systems do not recognize avian antibodies as useful for their abilities to recognize certain pathogens, or the specificities of avian antibodies for antigens of such pathogens, avian antibodies do not even elicit the desired immune responses in mammals.”

With varying degrees of success, the prior art methods and compositions discussed above attempt to address a number of recognized difficulties in the field of immunization, namely, (a) the cost and difficulty in immunizing mammalian (e.g. bovine) stock against specific pathogens, (b) the inability of avian antibodies to trigger the mammalian complement cascade, and (c) the tendency of mammalian immune systems to reject and develop antibodies against avian antibodies. It is clear that there exists a need for a simple and economical way to immunize mammalian subjects against specific pathogens in a manner that combines the benefits of passive and active immunity.

### SUMMARY OF INVENTION

The present invention involves the hyperimmunization of an avian population, such as chicken, Gallus domesticus, to produce specific antibodies in the eggs/egg yolks (antibodies maybe utilized with whole egg, egg yolk or extracted antibody applications), and production of a quantity of mammalian antibodies from non-immunized stock (mammalian antibodies may be provided in the form of commercial colostrum or serum products or extracted antibody isolated from colostrum, milk or blood serum). The avian and mammalian antibodies are combined to produce a composition that is effective in immunizing mammalian subjects, especially neonates, against pathogens, especially pathogens found in the digestive tract. The combination of the antibodies from hyperimmunized avian population with mammalian antibodies from non-immunized population has an unexpected synergistic immunological effect that is greater than the cumulative effects of administering avian and mammalian antibodies separately, sequentially or in isolation.

The composition and method of the invention are effective against a wide variety of antigens or combination of antigens. The antigens can be bacterial, viral, cellular, or any other substance to which the immune system of a domesticated fowl will respond, and which will induce a state of immune sensitivity in the fowl.

Hyperimmunization is known in the prior art. In the context of the present invention the avian source may be hyperimmunized by any one of a number of known methods. In particular, the present invention contemplates the hyperimmunization of the avian source by administration of vaccine adjuvants, such as such as Freund's adjuvant and mineral oil/water emulsions, in combination with immunogens and/or antigens, to dramatically increase the immune response. Any desired antigen may be used, however, antigens obtained from the following list of pathogens will be of particular relevance to the practice of the present invention: Salmonella, E. coli, Pasturella, Mycobacteria, Rotavirus, Coronavirus and any mixtures of bacterial antigens and viral antigens.

### DETAILED DESCRIPTION OF THE INVENTION

In this application immunization means a process that increases an organism's immunological reaction to an antigen by exposing the organism to the antigen, inducing a primary immune response, thereby improving its ability to resist or overcome infection by that antigen.

Hyperimmunization means a process whereby an organism is exposed to one or more antigens given repeatedly at high doses over time such that an immune response is dramatically elevated and maintained well above a routine vaccination schedule and markedly above a natural unexposed state.

Non-immunized refers to organisms that have not been recently exposed to a specified antigen (e.g. by immunization or hyperimmunization), such that the organisms do not exhibit elevated serum titers of antibody specific for that antigen. An organism that has been exposed to a specific antigen (e.g. immunized or hyperimmunized) may still be considered to be non-immunized with respect to other antigens or, it may be considered to be non-immunized with respect to that specific antigen if the organism does not exhibit elevated serum levels of antibodies specific to that antigen (vaccination failure). A group of organisms may be considered to be non-immunized with respect to a specific antigen if a majority of the organisms in the group do not exhibit elevated serum levels of antibodies specific to that antigen.

Specific antibody means an immunoglobulin molecule that has an amino acid sequence by virtue of which it interacts specifically with the antigen (or antigenic determinant) that induced its synthesis.

Viral Protection:

A blind study was conducted at a calf-raising facility that was experiencing a severe rotavirus infection in its calf population. In the study 15 holstein heifer calves were divided into three groups. For each group, sealed, color-coded pouches were prepared for each feeding. The technicians feeding and observing the calves had no knowledge of the pouch contents or the groupings.

The contents of the first feeding pouches were added to fresh bovine colostrum (non-immunized). The second feeding consisted of the contents of the second feeding pouch rehydrated in warm water.

Group I

Avian-sourced rotavirus antibody product and bovine colostrum (non-immunized source) were administered beginning day 0 in the first feeding after birth, avian-source rotavirus antibody in milk replacer was administered in the second feeding and in normal feedings twice a day for 14 days.

1^{st} feeding: 75 g bovine colostrum powder and 75 g avian-source rotavirus antibody in two quarts fresh maternal colostrum;

2^{nd} feeding: 204 g avian-sourced rotavirus antibody, 204 g milk replacer;

3^{rd} feeding to completion: milk replacer containing 10% avian-sourced rotavirus antibody product.

Group II

No avian-sourced rotavirus antibody product in the first two feedings. Avian rotavirus antibody administered beginning day 1 (3^{rd} feeding) in milk replacer fed twice daily for 14 days.

1^{st} feeding: 150 g bovine colostrum powder in two quarts fresh maternal colostrum;

2^{nd} feeding: 408 g milk replacer;

3^{rd} feeding to completion: milk replacer containing 10% avian-source rotavirus antibody product.

Group III (Control)

No avian rotavirus antibody product administered. Milk replacer fed twice daily for 14 days.

1^{st} feeding: 150 g bovine colostrum powder in two quarts fresh maternal colostrum;

2^{nd} feeding: 408 g milk replacer;

3^{rd} feeding to completion: 408 g milk replacer.

The avian-sourced rotavirus antibody product used in the study consisted of whole eggs taken from brown leghorn chickens immunized intramuscularly with a commercially available rotavirus vaccine. The whole eggs were heat-treated and spray dried. The finished product had a rotavirus titer of 10,240. The colostrum powder was a commercially available spray-dried colostrum product (non-immunized source). The milk replacer was Land-O-Lakes Cow's Match Milk Replacer™. Other equivalent products, which will be apparent to persons skilled in the art, may be used without departing from the scope of the invention.

This study was carried out during a rotavirus outbreak on a large dairy farm. So as not to introduce too many unknowns, for the 1^{st} feedings the farm was asked to feed normal maternal colostrum to all calves in all of the groups. All three groups also received additional powdered colostrum to ensure adequate passive transfer of antibodies. Blood tests on the animals following colostrum intake showed that all calves—including the Group III calves—received the minimum passive transfer level recommended by the USDA, 800 mg/dl.

The greatest reduction in rotavirus prevalence and duration occurred in Group I calves. The Group II calves showed a lesser degree of reduction in rotavirus. 60% of Group I calves tested positive for rotavirus, with an average duration of 1.0 days of infection. Group I calves had a total of 45% of fecal samples test positive for rotavirus days 7-10 after birth.

In Group II 80% of calves tested positive for rotavirus, with an average infection time of 1.6 days. 55% of group II samples tested were positive for rotavirus.

100% of group III (control) calves tested positive for rotavirus. 80% of all fecal samples tested in group III were positive for rotavirus, with an average duration of 2.2 days.

The 35% reduction in rotavirus infection duration/prevalence in group I as compared to group III is significant (p<0.05). Group II showed a 25% reduction as compared to group III.

| | | Number of | | | | | | | | |
|---|---|---|---|---|---|---|---|---|---|---|
| | | Calves | Percent of Calves | Average Duration | | | | | | |
| | | Positive for | positive for | of Rotavirus | No. of fecal samples positive/total on day | | | | | |
| Group | Feedings | rotavirus | rotavirus | Infection | 7 | 8 | 9 | 10 | Total | % |
| 1 | Avian Antibody | 3/5 | 60% | 1.0 days | 2/5 | 3/5 | 2/5 | 2/5 | 9/20 | 45% |
| | and Colostrum in | | | | | | | | | |
| | 1^{st} Feeding, | | | | | | | | | |
| | Avian Antibody | | | | | | | | | |
| | and Milk Replacer | | | | | | | | | |
| | in Subsequent | | | | | | | | | |
| | Feedings | | | | | | | | | |
| 2 | Colostrum only for | 4/5 | 80% | 1.6 days | 2/5 | 3/5 | 3/5 | 3/5 | 11/20 | 55% |
| | 1st Feeding, | | | | | | | | | |
| | No Colostrum and | | | | | | | | | |
| | No Avian Antibody | | | | | | | | | |
| | for 2^{nd} Feeding, | | | | | | | | | |
| | Avian Antibody for | | | | | | | | | |
| | Subsequent | | | | | | | | | |
| | Feedings | | | | | | | | | |
| 3 | Colostrum Only in | 5/5 | 100% | 2.2 days | 3/5 | 4/5 | 5/5 | 4/5 | 16/20 | 80% |
| | 1^{st} Feeding, | | | | | | | | | |
| | No Avian Antibody | | | | | | | | | |
| | and No Colostrum | | | | | | | | | |
| | afer 1^{st} feeding | | | | | | | | | |

All calves received adequate IgG transfer from colostrums regardless of treatment group. Many calves experienced fevers over the course of the study. Fevers of 103.5° F. or greater were treated with Banimine™ and/or Deliver™. Groups I and II had a delayed onset of fever (delayed by 2.3 days on average as compared to group III). Group I had 1.2 days less of fever on average than either groups II or III. Group I calves also had a 40% reduction in the need for antibiotic therapy compared to groups II and III.

In summary, group I calves, receiving avian-sourced antibody together with non-immunized colostrum in the first feeding, had on average less sever diarrhea, longer time periods before the onset of diarrhea, shorter duration and later onset of fever, 40% lower incidence of rotavirus and on average 35% less days with rotavirus shedding and a significant reduction in the need for antibiotic therapy compared to control calves. Group II calves, receiving only non-immunized colostrum in the first feeding and then receiving only avian-sourced rotavirus antibody in the third and subsequent feedings, had results intermediate groups I and III in terms of severity of diarrhea, onset of diarrhea, prevalence and duration of rotavirus infection. Calves in group II had 20% lower incidence of rotavirus and a reduction of 25% in the duration of infection as compared to group III.

No cows (i.e. the mothers of the Group I, II and III calves) were immunized, nor was the colostrum (either the dried or the maternal) assayed for rotavirus content. The maternal colostrum and the bovine colostrum powder simply had a “normal” complement of antibodies that would be expected to be produced by non-immunized animals.

Bacterial Challenge Protection:

Holstein neonatal calves were divided into two groups. The test composition was prepared from a blend of avian-sourced Salmonella typhimurium-specific antibody (in this case, egg powder), dried bovine colostrum powder (non-immunized), skim milk powder (i.e. dairy filler) and maltodextrin (i.e. flow agent). The colostrum powder is preferably a non-specific antibody product that is veterinary biologic registered with the USDA and CFIA for providing calves with passive transfer antibodies.
Group I: Control—Received 350 g of milk replacer at first feeding after birth. Second and subsequent feedings consisted of 350 g of milk replacer per twice daily feeding.
Group II: Test—Received one 350 g package of colostrum powder (e.g. First Start-50™, produced by LaBelle Inc.) at first feeding after birth. Second and subsequent feedings consisted of 30 g the test composition in 320 g milk replacer per twice daily feeding.

All calves were challenged with one dose of Salmonella typhimurium containing 10⁶ colony forming units no earlier than 36 hours after birth.

Results: There was 0% mortality in the Group II calves compared with 66% mortality in calves in Group I.

In mammalian neonates, colostrum is absorbed in order to protect the infant from infections until their own immune systems are fully functioning. Avian-sourced antibodies, from hyperimmunized laying hens, can provide a higher level of specificity to a potential disease outbreak. The combination of a concentrated level of antibodies from the avian source with the readily absorbable immunoglobulins from the mammalian source produces the synergistic effect of this invention. Avian antibodies provide the specificity necessary to attack the pathogen (e.g. virus, bacteria, parasite, etc. ) in the digestive tract and the mammalian-sourced antibodies bring the whole of the animal's immune system into play.

What is claimed is:

1. A product for the prevention and therapy of an infection in a mammal, wherein said mammal is a mammal of a given species, and wherein said infection is caused by a pathogen, said product comprising:
a) avian-sourced antibodies specific for said pathogen, wherein said avian-sourced antibodies are produced by avian animals immunized against said pathogen; and
b) mammalian antibodies, wherein said mammalian antibodies are produced by non-immunized mammals of said species.

2. The product of claim 1, wherein said product additionally comprises at least one of probiotics, vitamins and minerals.

3. The product of claim 1, wherein said mammal is a calf.

4. The product of claim 1, wherein said pathogen is one of rotavirus and salmonella typhimurium.

5. The product of claim 1, wherein said avian animals are chickens.

6. The product of claim 1, wherein said avian-sourced antibodies are in the form of egg, whole egg powder, egg yolk, egg yolk powder, egg extract, serum or serum extract.

7. The product of claim 1, wherein said mammalian antibodies are in the form of colostrum, colostrums powder, colostrums extract, serum or serum extract.

8. The product of claim 1, wherein said product is intended to be administered orally.

9. The product of claim 1, wherein said avian animals are hyperimmunized.

10. The product of claim 9, wherein said avian animals are hyperimmunized using at least one of:
a) an antigen obtained from Salmonella, E. coli, Pasturella, Mycobacteria, Rotavirus, or Coronavirus; and
b) a vaccine adjuvant for increasing an immune response, wherein said vaccine adjuvant is one of Freund's adjuvant and a mineral oil/water emulsion.

11. A method of prevention and therapy of an infection in a mammal, wherein said mammal is a mammal of a given species, and wherein said infection is caused by a pathogen, said method comprising:
a) administering to said mammal:
i) avian-sourced antibodies specific for said pathogen, wherein said avian-sourced antibodies are produced by avian animals immunized against said pathogen; and
ii) mammalian antibodies, wherein said mammalian antibodies are produced by non-immunized mammals of said species;
wherein said avian sourced antibodies and said mammalian antibodies are administered to said mammal at the same time.

12. The method of claim 11, further comprising administering to said mammal at least one of probiotics, vitamins and minerals.

13. The method of claim 11, wherein said mammal is a calf.

14. The method of claim 11, wherein said pathogen is one of rotavirus and salmonella typhimurium.

15. The method of claim 11, wherein said avian animals are chickens.

16. The method of claim 11, wherein said avian-sourced antibodies are in the form of egg, whole egg powder, egg yolk, egg yolk powder, egg extract, serum or serum extract.

17. The method of claim 11, wherein said mammalian antibodies are in the form of colostrum, colostrums powder, colostrums extract, serum or serum extract.

18. The method of claim 11, wherein said avian-sourced antibodies and said mammalian antibodies are administered orally.

19. The method of claim 11, wherein said avian animals are hyperimmunized.

20. The method of claim 11, wherein said avian-sourced antibodies and said mammalian antibodies are administered in the first feeding after birth.

21. The method of claim 19, wherein said avian animals are hyperimmunized using at least one of:
a) an antigen obtained from Salmonella, E. coli, Pasturella, Mycobacteria, Rotavirus, or Coronavirus; and
b) a vaccine adjuvant for increasing an immune response, wherein said vaccine adjuvant is one of Freund's adjuvant and a mineral oil/water emulsion.
